(19) United States
(12) Patent Application Publication
(10) Pub. No.: US 2014/0303631 A1
(43) Pub. Date: Oct. 9, 2014

(54) METHOD AND APPARATUS FOR DETERMINING THE ORIENTATION AND/OR POSITION OF AN OBJECT DURING A MEDICAL PROCEDURE

(71) Applicant: Thornberry Technologies, LLC, Tallahassee, FL (US)

(72) Inventor: Robert L. Thornberry, Tallahassee, FL (US)

(21) Appl. No.: 14/247,159

(22) Filed: Apr. 7, 2014

Related U.S. Application Data
(60) Provisional application No. 61/809,111, filed on Apr. 5, 2013.
(60) Provisional application No. 61/874,534, filed on Sep. 6, 2013.

(51) Int. Cl. A61F 2/46

(52) U.S. Cl. CPC A61F 2/4609; A61F 2002/4632; A61F 2002/4668
USPC 606/91

(57) ABSTRACT

A computer-guided system for determining the disposition of an object, the computer-guided system comprising:
a platform;
a compass removably and adjustably mounted to the platform, the compass comprising:
   a first arm having a proximal end and a distal end;
   a second arm having a proximal end and a distal end;
   the proximal end of the first arm being removably and adjustably mounted to the platform by a magnetic ball mount, wherein the magnetic ball mount comprises a spherical encoder;
   the proximal end of the second arm being movably mounted to the distal end of the first arm by a pivot mount, wherein the pivot mount comprises an angular sensor; and
determining means for determining the disposition of the distal end of the second arm relative to the platform by using data from the spherical encoder and the angular sensor.

### REFERENCE TO PENDING PRIOR PATENT APPLICATIONS

This patent application claims benefit of:

(i) pending prior U.S. Provisional Patent Application Ser. No. 61/809,111, filed Apr. 5, 2013 by Robert L. Thornberry for COMPUTER-GUIDED SYSTEM FOR ORIENTING THE ACETABULAR CUP IN THE PELVIS DURING TOTAL HIP REPLACEMENT SURGERY (Attorney's Docket No. THORNBERRY-9 PROV); and

(ii) pending prior U.S. Provisional Patent Application Ser. No. 61/874,534, filed Sep. 6, 2013 by Robert L. Thornberry for METHOD AND APPARATUS FOR JOINT SURGERY (Attorney's Docket No. THORNBERRY-10 PROV).

The two (2) above-identified patent applications is hereby incorporated herein by reference.

### FIELD OF THE INVENTION

This invention relates to computer-guided surgery in general, and more particularly to methods and apparatus for determining the orientation and/or position of an object during a medical procedure, including methods and apparatus for orienting the acetabular cup in the acetabulum during total hip replacement surgery.

### BACKGROUND OF THE INVENTION

Joint replacement surgery seeks to replace portions of a joint with prosthetic components so as to provide long-lasting joint function and pain-free mobility for the patient.

One joint which is commonly replaced, in whole or in part, is the hip joint. The hip joint is located at the junction of the femur and the acetabulum. More particularly, and looking now at FIG. 1, the head of the femur is received in the acetabulum, with a plurality of ligaments and other soft tissue serving to hold the bones in articulating relation.

During total hip replacement surgery, and looking now at FIG. 2, the operative elements of the hip joint (i.e., the head of the femur and the acetabular cup) are replaced by prosthetic components. More particularly, during total hip replacement surgery, the head of the femur is replaced by a prosthetic ball-and-stem and the native acetabular cup is replaced by a prosthetic acetabular cup, whereby to provide the prosthetic total hip joint.

The present invention will hereinafter be discussed in the context of a total hip replacement surgery, however, it should also be appreciated that the present invention may be equally applicable to other types of hip surgery where components of the hip need to be replaced, and/or to other joint replacement surgery.

In order to replace the head of the femur with the femoral prosthesis, the head of the femur is first distracted from the acetabulum so as to expose the femoral head. Then an osteotomy is performed on the femoral neck so as to remove the neck and head of the femur from the remainder of the femur. Next, the proximal end of the intramedullary canal is prepared to receive the stem of the femoral prosthesis. More particularly, a rasp, reamer, broach, etc. is used to hollow out, clean and enlarge the intramedullary canal of the femur so as to create a cavity to receive the stem of the femoral prosthesis. Once the proximal end of the intramedullary canal has been prepared to receive the femoral prosthesis, the stem of the femoral prosthesis is inserted into the intramedullary canal so that the ball of the femoral prosthesis is appropriately presented to the acetabular cup. Typically, the ball of the femoral prosthesis is formed separately from the stem of the femoral prosthesis, and it is mounted to the stem of the femoral prosthesis at the time of use. Furthermore, it should also be appreciated that during the surgery itself, it is common to temporarily position a trial stem or broach in the femur, attach a trial ball or equivalent element to the trial stem or broach, and then temporarily reduce the joint so as to confirm the reconstruction before the actual prosthetic stem is secured in position within the femur.

In order to replace the native acetabular cup with the prosthetic acetabular cup, the native acetabulum is first prepared to receive the prosthetic acetabular cup. This generally involves reaming an appropriate seat in the acetabulum to receive the prosthetic acetabular cup. Then the prosthetic acetabular cup is installed in the seat formed in the acetabulum, and the distraction released, so that the ball of the femoral prosthesis can be seated in the prosthetic acetabular cup. In this respect it will be appreciated that the prosthetic acetabular cup typically comprises an outer cup made of metal and an inner liner made of polyethylene (or another polymer, or a ceramic, or a metal, etc.). The metal outer cup is configured so as to be received in the seat formed in the acetabulum and thereafter osseointegrate into the host bone, and the polyethylene inner liner is configured so as to be received in the metal outer cup and thereafter provide a low-friction seat for the ball of the femoral prosthesis.

During seating of the prosthetic acetabular cup in the acetabulum, it is important that the prosthetic acetabular cup be set in the acetabulum with the proper positioning, i.e., at the proper location and with the proper orientation. Such proper positioning is important in order to (i) avoid impingement between the rim of the prosthetic acetabular cup and the neck of the femoral prosthesis as the prosthetic joint is moved through a range of motions, since such impingement can result in a reduced range of motion, excessive wear, joint failure and/or substantial pain for the patient, and (ii) avoid dislocation of the ball of the femoral prosthesis from the acetabular cup as the joint is moved through a range of motions, since such dislocation can result in damage to the anatomy, joint failure and/or substantial pain for the patient.

In many cases, the surgeon seats the prosthetic acetabular cup in the acetabulum “by eye”, and thereafter confirms the proper disposition of the prosthetic acetabular cup when the distracted joint is subsequently reduced. However, this approach relies heavily on the anatomical view available to, and appreciated by, the surgeon, and errors in cup orientation (i.e., tilt) may not be discovered until after the surgery has been completed, since such errors in cup orientation can be difficult to detect interoperatively, even where X-ray imaging is available.

For this reason, various computer-guided systems have been developed to assist the surgeon in the proper placement of the prosthetic acetabular cup during total hip replacement surgery. However, such computer-guided systems frequently require that a CT scan be made of the patient in advance of the procedure so as to determine the geometry of the acetabulum. Furthermore, such computer-guided systems typically require (i) the registration and tracking of pelvic anatomical landmarks (e.g., the anterior/superior iliac spines, which are sometimes referred to as the “ASIS” points, and the pubic tubercles, which are sometimes referred to as the “PTUB” points) prior to and during the surgery, e.g., with optical or electromagnetic trackers placed on the pelvic anatomical landmarks, and (ii) the registration and tracking of femoral anatomical landmarks prior to and during the surgery, e.g., with optical or electromagnetic trackers placed on the femoral anatomical landmarks. However, in practice, one or more of the pelvic anatomical landmarks can be difficult to physically access during the procedure. Furthermore, the optical or electromagnetic trackers must typically be applied to both the pelvic anatomical landmarks and the femoral anatomical landmarks during the surgery itself so as to track the dispositions of these body parts during the surgery. These requirements can add to the cost of the procedure, can lengthen the time required for the procedure, and can be inconvenient for the surgeon (e.g., such as where the surgeon must work around optical trackers protruding into the surgical field). In this respect it should be appreciated that optical trackers, while providing good spatial resolution, suffer from the disadvantage that they must remain directly visible at all times; electromagnetic trackers, while not requiring direct visual access, suffer from the disadvantage of poor spatial resolution. In addition, it should be appreciated that the optical or electromagnetic trackers are typically attached to the pelvis and/or femur using pins, which cause trauma to the bone.

Accordingly, there is a need for a new and improved computer-guided system for orienting a prosthetic acetabular cup in the acetabulum during total hip replacement surgery, wherein the need for a pre-operative CT scan can be eliminated, and wherein the need to physically access pelvic anatomical landmarks during the procedure can be eliminated (and the need to attach optical or electromagnetic trackers using pins can be eliminated).

In addition, there is also a need for a new and improved computer-guided system which can be used to orient prosthetic components other than a prosthetic acetabular cup, e.g., a computer-guided system which can be used to orient a femoral prosthetic component.

Furthermore, there is also a need for a new and improved computer-guided system which can be used to orient prosthetic components for joints other than the hip, e.g., a computer-guided system which can be used to orient prosthetic components in the knee.

And there is a need for a new and improved computer-guided system which can be used to orient substantially any two interacting components in space.

And there is a need for a new and improved computer-guided system which can be used to determine the orientation and/or position of an object during a medical procedure.

### SUMMARY OF THE INVENTION

The present invention provides a new and improved computer-guided system for orienting a prosthetic acetabular cup in the acetabulum during total hip replacement surgery, wherein the need for a pre-operative CT scan can be eliminated, and wherein the need to physically access pelvic anatomical landmarks during the procedure can be eliminated (and the need to attach optical or electromagnetic trackers using pins can be eliminated).

In addition, the present invention provides a new and improved computer-guided system which can be used to orient prosthetic components other than a prosthetic acetabular cup, e.g., a computer-guided system which can be used to orient a femoral prosthetic component.

Furthermore, the present invention provides a new and improved computer-guided system which can be used to orient prosthetic components for joints other than the hip, e.g., a computer-guided system which can be used to orient prosthetic components in the knee.

And the present invention provides a new and improved computer-guided system which can be used to orient substantially any two interacting components in space.

And the present invention provides a new and improved computer-guided system which can be used to determine the orientation and/or position of an object during a medical procedure.

In one preferred form of the invention, there is provided a computer-guided system for determining the disposition of an object, the computer-guided system comprising:

a platform;

a compass removably and adjustably mounted to the platform, the compass comprising:
a first arm having a proximal end and a distal end;
a second arm having a proximal end and a distal end;
the proximal end of the first arm being removably and adjustably mounted to the platform by a magnetic ball mount, wherein the magnetic ball mount comprises a spherical encoder;
the proximal end of the second arm being movably mounted to the distal end of the first arm by a pivot mount, wherein the pivot mount comprises an angular sensor; and

determining means for determining the disposition of the distal end of the second arm relative to the platform by using data from the spherical encoder and the angular sensor.

In another preferred form of the invention, there is provided

In another preferred form of the invention, there is provided a method for determining the disposition of an object, the method comprising:

providing a computer-guided system for determining the disposition of an object, the computer-guided system comprising:
a platform;
a compass removably and adjustably mounted to the platform, the compass comprising:
   a first arm having a proximal end and a distal end;
   a second arm having a proximal end and a distal end;
   the proximal end of the first arm being removably and adjustably mounted to the platform by a magnetic ball mount, wherein the magnetic ball mount comprises a spherical encoder;
   the proximal end of the second arm being movably mounted to the distal end of the first arm by a pivot mount, wherein the pivot mount comprises an angular sensor; and
determining means for determining the disposition of the distal end of the second arm relative to the platform by using data from the spherical encoder and the angular sensor;

mounting the object to the distal end of the second arm; and

using the computer-guided system to determine the orientation of the object relative to the platform.

In another preferred form of the invention, there is provided a method for setting a prosthetic acetabular cup in the native acetabular cup with a desired inclination and anteversion, the method comprising:

providing a computer-guided system for determining the orientation of the prosthetic acetabular cup, the computer-guided system comprising:
a platform;
a compass removably and adjustably mounted to the platform, the compass comprising:
   a first arm having a proximal end and a distal end;
   a second arm having a proximal end and a distal end;
   the proximal end of the first arm being removably and adjustably mounted to the platform by a magnetic ball mount, wherein the magnetic ball mount comprises a spherical encoder;
   the proximal end of the second arm being movably mounted to the distal end of the first arm by a pivot mount, wherein the pivot mount comprises an angular sensor; and
determining means for determining the disposition of the distal end of the second arm relative to the platform by using data from the spherical encoder and the angular sensor;

determining the two ASIS points;

determining the center of the hip using the computer-guided system;

determining the HCAPP using the two ASIS points and the center of the hip;

determining the calculated APP using the HCAPP;

mounting the prosthetic acetabular cup to the distal end of the second arm; and

using the computer-guided system to set the prosthetic acetabular cup in the native acetabular cup with a desired inclination and anteversion.

In another preferred form of the invention, there is provided a computer-guided system for determining the disposition of an object, the computer-guided system comprising:

a platform;

an object;

a first compass removably and adjustably mounted to the platform, the first compass comprising: a first arm having a proximal end and a distal end; a second arm having a proximal end and a distal end; the proximal end of the first arm being removably and adjustably mounted to the platform by a magnetic ball mount; the proximal end of the second arm being movably mounted to the distal end of the first arm by a pivot mount, wherein the pivot mount comprises an angular sensor; an accelerometer mounted to at least one of the first arm and the second arm; wherein the distal end of the second arm is mounted to the object;

a second compass removably and adjustably mounted to the platform, the second compass comprising: a first arm having a proximal end and a distal end; a second arm having a proximal end and a distal end; the proximal end of the first arm being removably and adjustably mounted to the platform by a magnetic ball mount; the proximal end of the second arm being movably mounted to the distal end of the first arm by a pivot mount, wherein the pivot mount comprises an angular sensor; an accelerometer mounted to at least one of the first arm and the second arm; wherein the distal end of the second arm is mounted to the object; and

determining means for determining the disposition of the relative to the platform by using data from the angular sensor and accelerometer of the first compass and data from the angular sensor and accelerometer of the second compass.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing skeletal anatomy in the area of the hip joint;

FIG. 2 is a schematic view showing a total hip replacement;

FIGS. 3 and 4 are schematic views showing the anterior pelvic plane (APP);

FIG. 5 is a schematic view showing the hip center anterior superior spine plane (HCAPP)

FIGS. 6-8 are schematic views showing a novel computer-guided system formed in accordance with the present invention;

FIG. 9 is a schematic view showing a spherical encoder used in the novel computer-guided system of FIGS. 6-8;

FIG. 10 is a schematic view showing how various components of the novel computer-guided system of FIGS. 6-8 are connected together;

FIG. 11 is a schematic view showing how the spherical ferrous metal ball of an impactor of the novel computer-guided system of FIGS. 6-8 will follow a hemispherical orbit as the impactor is moved about; and

FIG. 12 is a flowchart illustrating operation of the present invention.

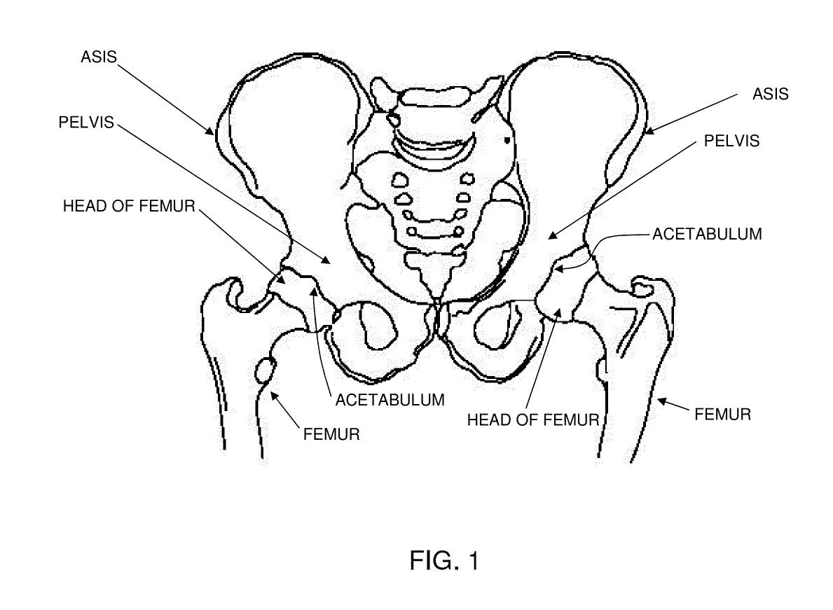

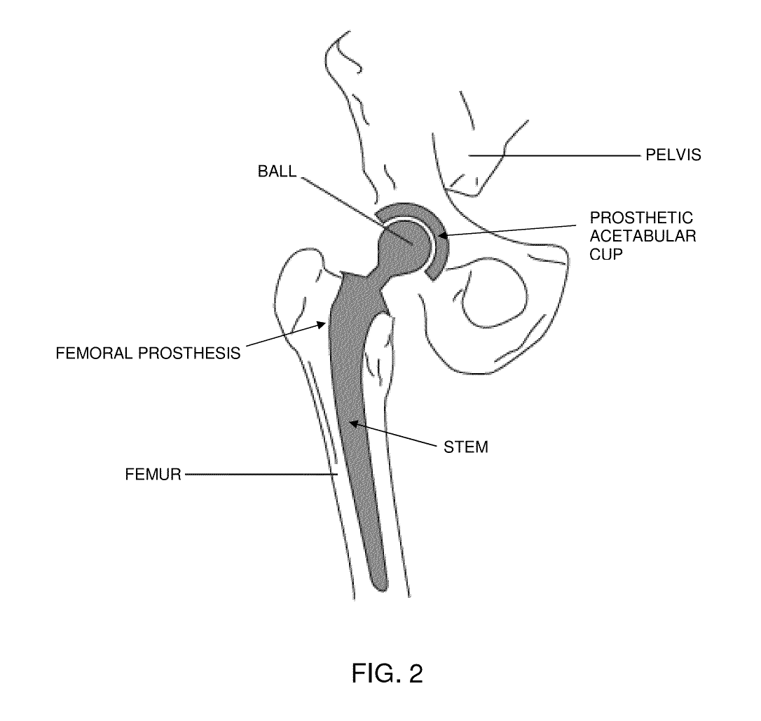

### DETAILED DESCRIPTION OF THE PREFERRED EMBODIMENTS

### Computer-Guided System for Providing Improved Accuracy in the Placement of a Prosthetic Acetabular Cup During Total Hip Replacement Surgery

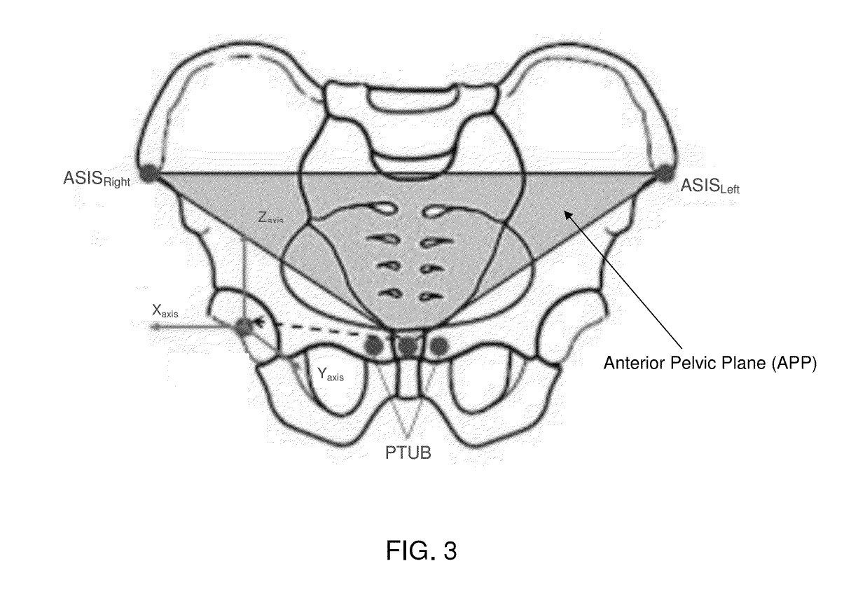

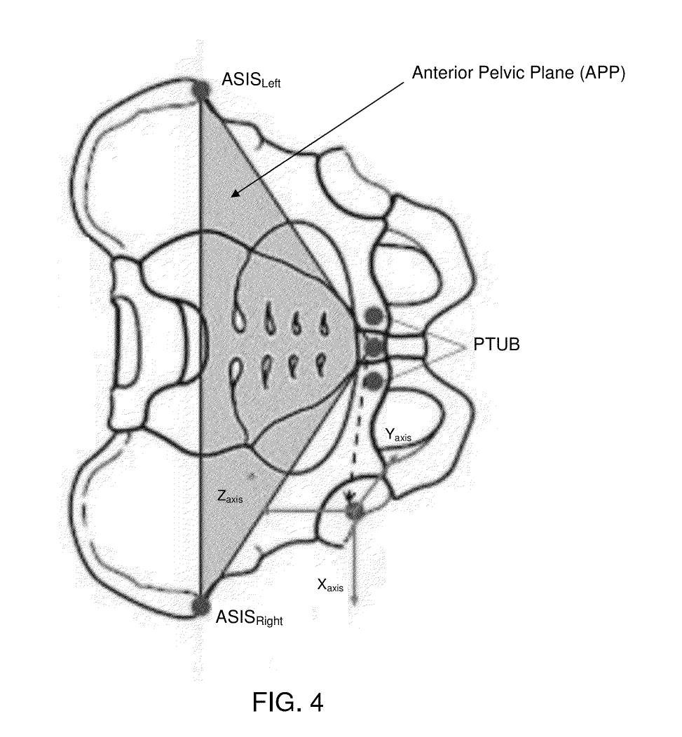

Looking now at FIGS. 3 and 4, surgeons typically orientate a prosthetic acetabular cup by identifying the proper placement of the acetabular cup vis-à-vis the pelvis, by specifying the desired degree of inclination and the desired degree of anteversion for the acetabular cup with respect to the anterior pelvic plane (APP). The anterior pelvic plane (APP) is the plane defined by connecting three points: the two anterior/superior iliac spines (ASIS), each of which are located on opposite sides of the pelvis, and the pubic tubercles (PTUB).

Unfortunately, during surgery, the three points (ASIS, ASIS and PTUB) are not readily exposed for touching with a digitizer (e.g., they are typically concealed under a drape, disposed behind other anatomy, etc.). In addition, the pubic tubercles (PTUBs) are relatively poorly defined structures and hence introduce inaccuracies into the process.

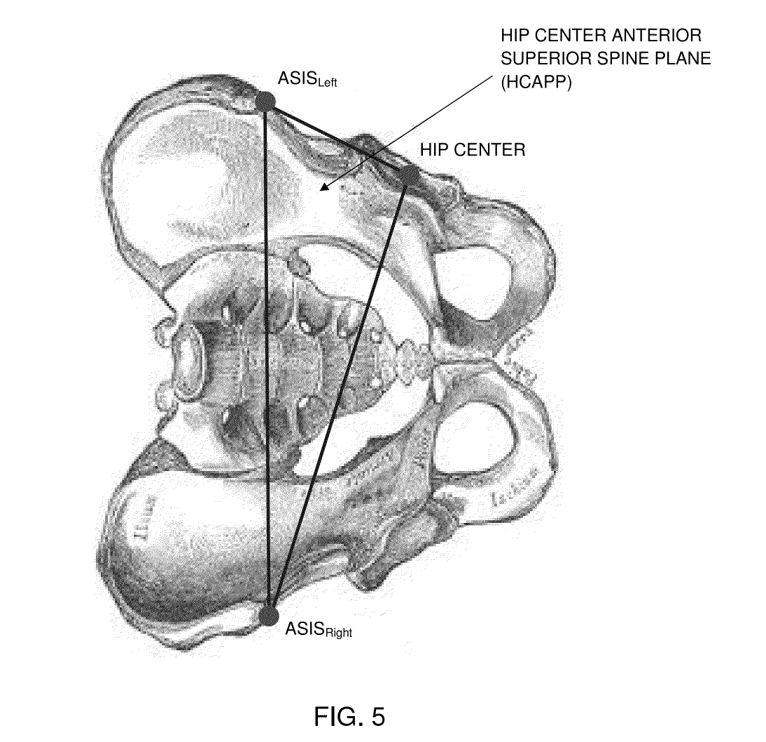

In view of the foregoing, and in accordance with the present invention, a different plane (i.e., one other than the anterior pelvic plane) is used to orient the prosthetic acetabular cup. More particularly, the present invention utilizes the plane defined by three points, i.e., the two ASIS points and the center of the hip. This plane is sometimes referred to herein as the hip center anterior superior spine plane (HCAPP), and is shown in FIG. 5. With the present invention, the hip center anterior superior spine plane (HCAPP) is used instead of the anterior pelvic plane (APP) for orienting a prosthetic acetabular cup. This is possible because general anatomical studies show that the HCAPP and the APP have a known relationship to one another, i.e., the HCAPP is generally set at a known angle relative to the APP. Thus, by identifying the HCAPP, the surgeon (or the computer-guided system) can calculate the calculated anterior pelvic plane (APP), and hence the surgeon can orient the prosthetic acetabular cup relative to the pelvis using the desired inclination and anteversion descriptors normally associated with the anterior pelvic plane (APP), e.g., “42 degrees inclination, 20 degrees anteversion”.

Accordingly, the present invention provides a novel method and apparatus for easily and accurately locating the center of the hip, so as to allow the surgeon to easily and accurately locate the hip center anterior superior spine plane (HCAPP), and hence allow the surgeon to easily and accurately locate the calculated anterior pelvic plane (APP). This then allows the surgeon to easily and accurately orient the prosthetic acetabular cup relative to the pelvis using the desired inclination and anteversion descriptors normally associated with the anterior pelvic plane (APP), e.g., “42 degrees inclination, 20 degrees anteversion”.

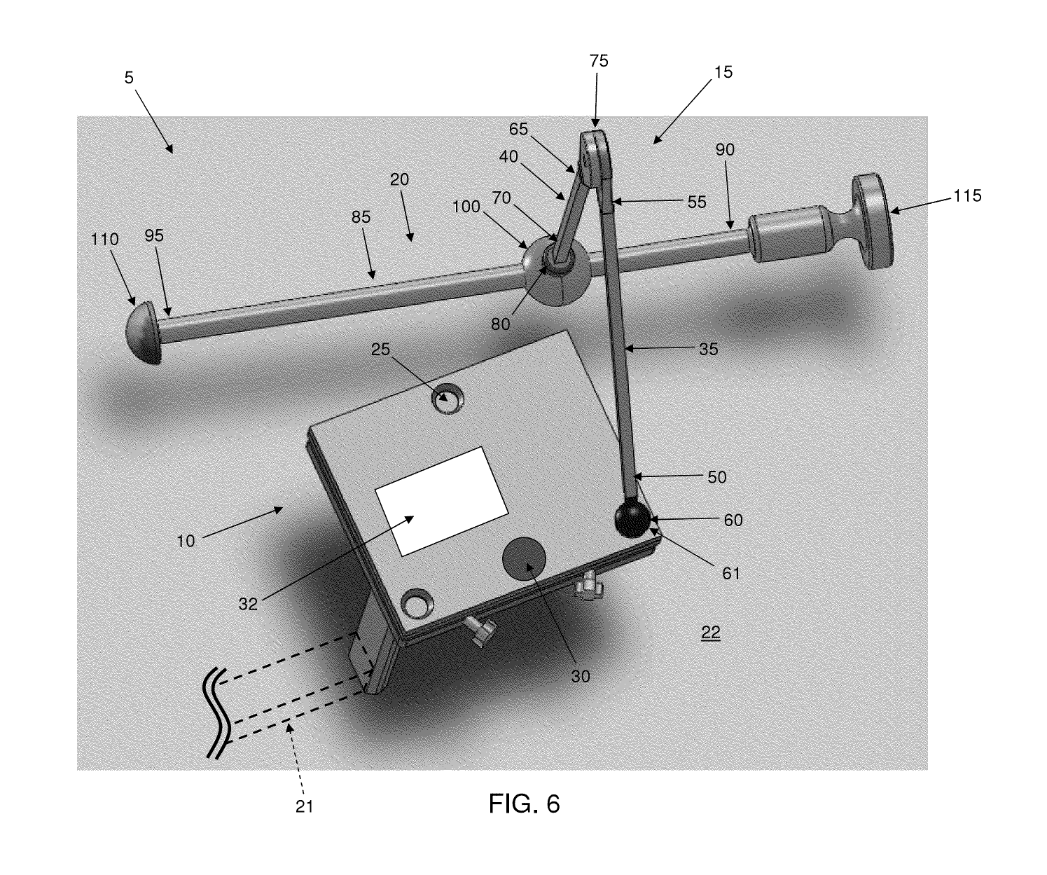

In one preferred form of the present invention, and looking now at FIGS. 6-8, there is provided a new and improved computer-guided system 5 which can be used to accurately locate the center of the hip. Computer-guided system 5 preferably comprises a platform 10 and a compass 15 which extends between platform 10 and an impactor 20.

Platform 10 is fixed to a patient positioner 21 (shown schematically in FIG. 6) which is, in turn, mounted to the operating table 22 (the top surface of which is shown schematically in FIG. 6). In one preferred form of the present invention, platform 10 comprises at least one magnetic spherical recess 25 for receiving a spherical ferrous metal ball set at one end of compass 15, as will hereinafter be discussed. In one form of the present invention, platform 10 comprises a plurality of magnetic spherical recesses 25. Platform 10 preferably also comprises a calibration hemisphere 30 for receiving the second end of compass 15 when calibrating computer-guided system 5, as will hereinafter be discussed. In one preferred form of the invention, platform 10 houses substantially all of the “smart” electronics of computer-guided system 5 (e.g., an appropriately-programmed computer, etc.), and includes a touchscreen 32 for allowing the surgeon to input data into computer-guided system 5 and/or to receive data from computer-guided system 5.

Compass 15 comprises a first arm 35 which is removably, adjustably mounted to platform 10, and a second arm 40 which is pivotally mounted to first arm 35. Second arm 40 removably receives impactor 20, as will hereinafter be discussed in further detail.

First arm 35 comprises a proximal end 50 and a distal end 55. A spherical ferrous metal ball 60 is fixed to proximal end 50 of first arm 35. Spherical ferrous metal ball 60 is sized to be received in a magnetic spherical recess 25 formed in platform 10, whereby to form a magnetic ball mount 61 which permits first arm 35 to be removably, adjustably mounted to platform 10 using magnetic forces.

In one preferred form of the present invention, magnetic spherical recess 25 utilizes so-called “rare earth” magnets so that substantial magnetic forces can be generated, whereby to provide a stable magnetic ball mount 61 for mounting compass 15 to platform 10. More particularly, sufficient magnetic forces are generated by the stable magnetic ball mount 61 so as to hold first arm 35 in a stable position relative to spherical recess 25 in platform 10; however, these magnetic forces may be manually overcome by a user so as to allow the position of first arm 35 to be moved to another position, whereupon the stable magnetic ball mount 61 will once again hold first arm 35 in a stable position relative to spherical recess 25 in platform 10. Thus, stable magnetic ball mount 61 essentially comprises a detachable mount for adjustably connecting compass 15 to platform 10.

Note that while in one preferred form of the invention stable magnetic ball mount 61 comprises a magnetic spherical recess 25 formed in platform 10 and a spherical ferrous metal ball 60 fixed to proximal end 50 of first arm 35, the arrangement could be reversed, i.e., stable magnetic ball mount 61 could comprise a ferrous metal spherical recess 25 formed in platform 10 and a spherical ferrous metal ball 60 fixed to proximal end 50 of first arm 35.

It should also be appreciated that while stable magnetic ball mount 61 has spherical ferrous metal ball 60 fixed to first arm 35 and spherical recess 25 formed in platform 10, the disposition of these components could be reversed, e.g., spherical ferrous metal ball 60 could be mounted to platform 10 and spherical recess 25 could be formed on proximal end 50 of first arm 35.

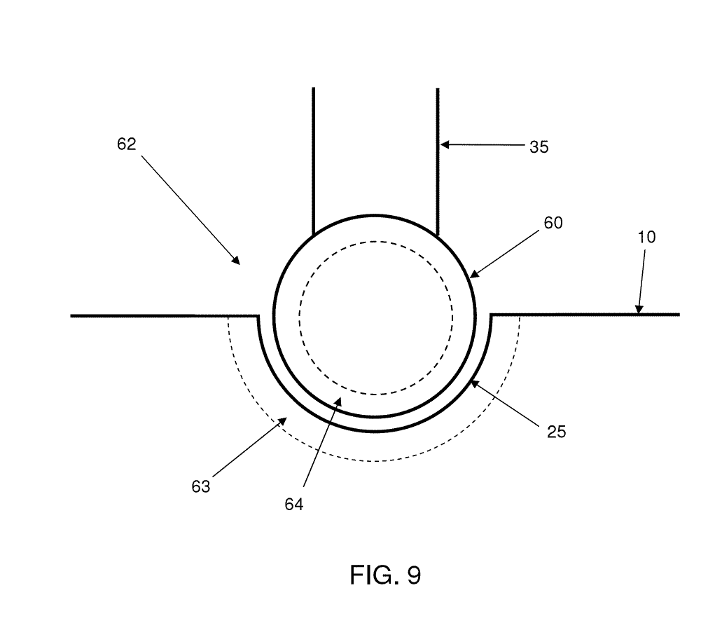

In one preferred form of the present invention, and looking now at FIG. 9, magnetic spherical recess 25 and spherical ferrous metal ball 60 together comprise a spherical encoder 62 (e.g., an optical spherical encoder, a magnetic spherical encoder, a mechanical spherical encoder, etc.) of the sort well known in the art (e.g., utilizing a sensor 63 for sensing disposition of a sensed portion 64) which is capable of determining and reporting the disposition of first arm 35 of compass 15 relative to platform 10.

Second arm 40 comprises a proximal end 65 and a distal end 70. Proximal end 65 of second arm 40 is pivotally mounted to distal end 55 of first arm 35 via a one degree-of-freedom hinged angular joint 75. Angular joint 75 is sufficiently “tight” such that first arm 35 and second arm 40 will normally maintain a given disposition relative to one another, however, this disposition may be manually overcome by a user so as to allow first arm 35 to be repositioned relative to second arm 40. Angular joint 75 preferably comprises an angular sensor 76 for determining the disposition of the angular joint (i.e., the angular disposition of first arm 35 relative to second arm 40). Angular sensor 76 may comprise any one of the many angular sensors well-known in the art, e.g., an optical angle reader, a magnetic angle reader, etc. Distal end 70 of second arm 40 comprises a concave magnetic mount 80 for magnetically attaching impactor 20 to compass 15, as will hereinafter be discussed.

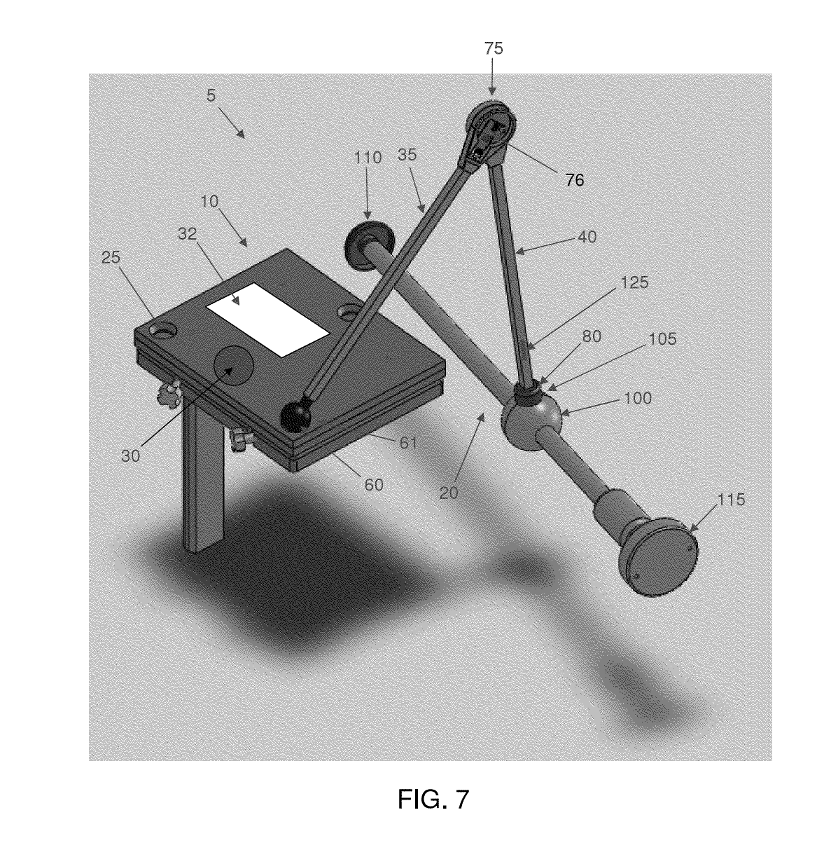

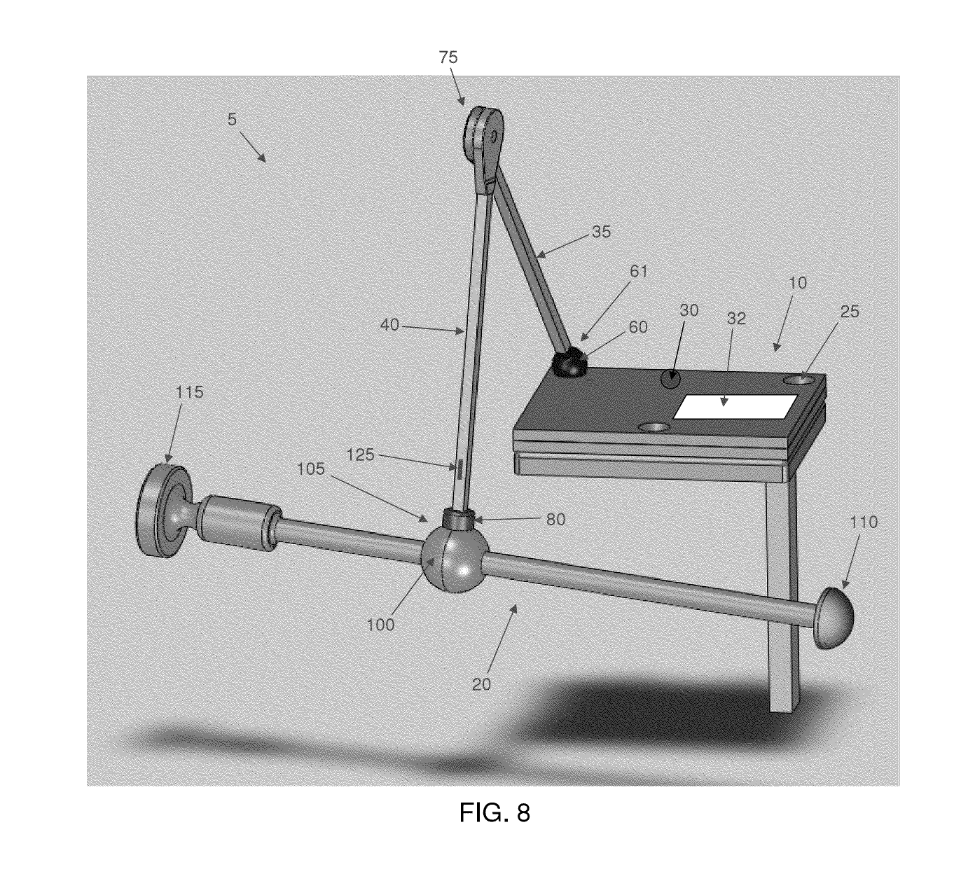

Impactor 20 comprises a shaft 85 having a proximal end 90, a distal end 95 and a spherical ferrous metal ball 100 disposed on shaft 85. Spherical ferrous metal ball 100 of impactor 20 is magnetically received by concave magnetic mount 80 of second arm 40, whereby to provide a stable magnetic ball mount 105 (FIGS. 7 and 8) for mounting impactor 20 to second arm 40 such that the longitudinal axis of second arm 40 is disposed perpendicular to the outer surface of the spherical ferrous metal ball 100. More particularly, sufficient magnetic forces are generated by the concave magnetic mount 80 so as to hold spherical ferrous metal ball 100 in a stable position relative to concave magnetic mount 80; however, these magnetic forces may be manually overcome by a user so as to allow the position of impactor 20 to be moved to another position, whereupon the concave magnetic mount 80 will once again hold spherical ferrous metal ball 100 in a stable position relative to concave magnetic mount 80. Thus, stable magnetic ball mount 105 essentially comprises a detachable mount for adjustably connecting impactor 20 to compass 15.

Note that while in one preferred form of the invention stable magnetic ball mount 105 comprises a concave magnetic mount 80 at the distal end of second arm 40 and a spherical ferrous metal ball 100 fixed to shaft 85 of impactor 20, the arrangement could be reversed, i.e., stable magnetic ball mount 105 could comprise a concave ferrous metal mount 80 at the distal end of second arm 40 and a magnetic spherical ball 100 fixed to shaft 85 of impactor 20.

A prosthetic acetabular cup 110, sized to be received in the native hip joint, is releasably mounted to the distal end of shaft 85 of impactor 20. A strike plate 115 is fixed to proximal end 90 of shaft 85 of impactor 20, whereby to provide a surface for the surgeon to strike with a mallet, so as to permit the surgeon to set prosthetic acetabular cup 110 in ways well known in the art.

Note that while the figures show impactor 20 having a straight shaft 85, impactor 20 may also be angled. In this situation, spherical ferrous metal ball 100 is positioned so that the center of spherical ferrous metal ball 100 is disposed along the line of impaction.

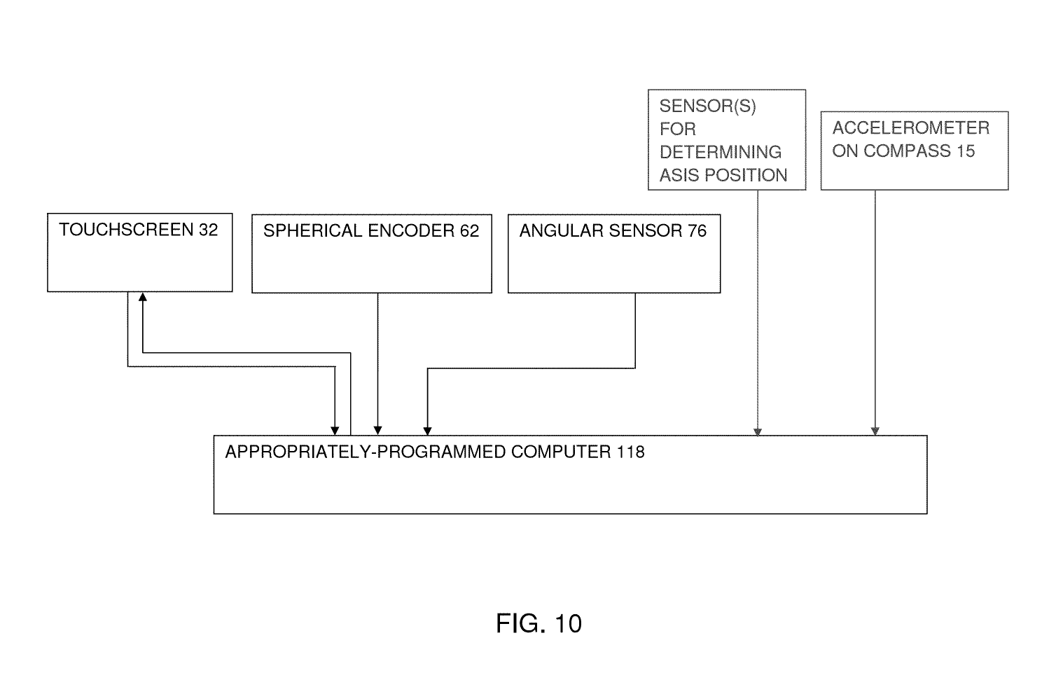

As seen in FIG. 10, computer-guided system 5 has its touchscreen 32, spherical encoder 62 and angular sensor 76 connected to the “smart” electronics of computer-guided system 5, e.g., to an appropriately-programmed computer 118. In addition, appropriately-programmed computer 118 is also connected to the sensor(s) which are used to determine the position of the ASIS points. Furthermore, if an additional spherical encoder 62 is provided on the distal end of compass 15 (see below), the additional spherical encoder 62 is also connected to appropriately-programmed computer 118. In addition, if an accelerometer 125 is provided on compass 15 (see below), the accelerometer 125 is also connected to appropriately-programmed computer 118. As noted above, the “smart” electronics of computer-guided system 5 (e.g., appropriately-programmed computer 118) are preferably housed in, or below, or on, platform 10. Touchscreen 32, spherical encoder 62 and angular sensor 76 are preferably connected to appropriately-programmed computer 118 via a wireless link (e.g., a Bluetooth link, etc.), although wires may be used if desired.

In one preferred form of the invention, a plastic sheet or cover (not shown) is positioned over platform 10 (which contains the electronics, touch screen, etc. of computer-guided system 5) prior to mounting first arm 35 to platform 10 using the aforementioned magnetic ball mount 61. In this way, the electronics of computer-guided system 5 remain outside of the sterile surgical field, and only compass 15 enters the sterile surgical field, with the aforementioned plastic sheet or cover demarcating the boundary of the sterile surgical field (and thereby separating the electronics of computer-guided system 5 from the sterile surgical field).

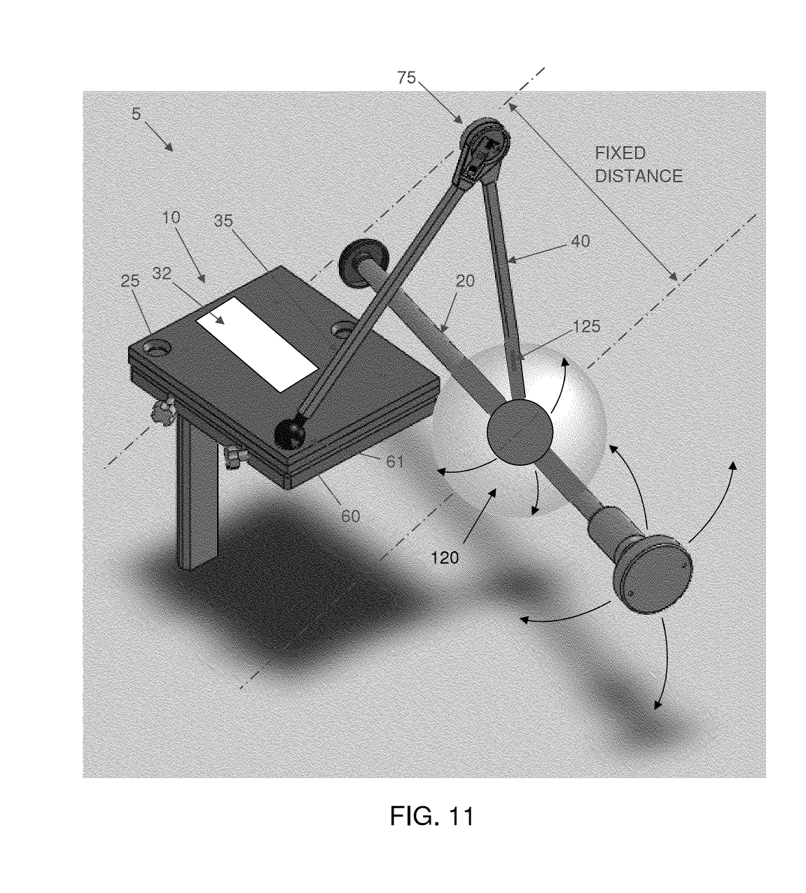

The surgeon uses computer-guided system 5 to identify the center of the hip joint by mounting compass 15 to platform 10 (i.e., by mounting first arm 35 to platform 10 using the aforementioned magnetic ball mount 61), by mounting impactor 20 (with prosthetic acetabular cup 110 attached to the distal end thereof) to compass 15 (i.e., by mounting impactor 20 to second arm 40 using the aforementioned stable magnetic ball mount 105), by moving impactor 20 so as to position the prosthetic acetabular cup 110 in the native acetabular cup, and then moving shaft 85 of impactor 20 about, while keeping prosthetic cup 110 in the native acetabular cup. See FIG. 11. Note that stable magnetic ball mount 61, angular joint 75 and stable magnetic ball mount 105 all articulate to the extent necessary to accommodate this movement. This movement of shaft 85 of impactor 20 about as the prosthetic acetabular cup remains in the native acetabular cup causes spherical ferrous metal ball 100 of impactor 20 (and hence, distal end 70 of second arm 40) to follow a hemispherical orbit 120 about the native acetabular cup (in this respect, note that ferrous metal ball 100 is set a fixed distance from the distal end of impactor 20). As this occurs, computer-guided system 5 uses the data reported by the spherical encoder 62 located at the magnetic ball mount 61 to determine the disposition of first arm 35 of compass 15, and the data reported by the angular sensor 76 located at the intersection of first arm 35 and second arm 40, to determine the changing position of spherical ferrous metal ball 100 of impactor 20, whereby to determine the hemispherical orbit 120 followed by spherical ferrous metal ball 100 about the native acetabular cup, whereby to solve for the center of the hemispherical orbit followed by spherical ferrous metal ball 100, and hence identify the center of the hip joint. Note that impactor 20 only needs to move about enough to generate a sufficient number of data points to solve for the center of the hemispherical orbit followed by spherical ferrous metal ball 100 and hence identify the center of the hip joint. In practice, this can be achieved by moving spherical ferrous metal ball 100 of impactor 20 about for a few seconds.

Having thus found the center of the hip joint, and having previously found the two ASIS points (which can be reported to computer-guided system 5), computer-guided system 5 can then calculate the hip center anterior superior spine plane (HCAPP), and hence the calculated anterior pelvic plane (APP). And, significantly, computer-guided system 5 can then display the current disposition of impactor 20 relative to the calculated anterior pelvic plane (APP), with the current disposition of impactor 20 being displayed in a digital readout on touchscreen 32 showing the current inclination and anteversion of impactor 20 in the context of inclination and anteversion to the calculated anterior pelvic plane (APP).

The surgeon can then use computer-guided system 5 to guide prosthetic acetabular cup 110 into the hip joint using the desired inclination and anteversion descriptors normally associated with the anterior pelvic plane (APP), e.g., “42 degrees inclination, 20 degrees anteversion”. Specifically, while watching the readout on touchscreen 32 on platform 10, the surgeon uses impactor 20 to precisely and accurately control the angular disposition of prosthetic acetabular cup 110 relative to the calculated anterior pelvic plane (APP). When prosthetic acetabular cup 110 is accurately aligned with the desired orientation for implantation (e.g., when touchscreen 32 reads “42 degrees inclination, 20 degrees anteversion”, or some other desired orientation for the prosthetic acetabular cup 110 vis-à-vis the calculated anterior pelvic plane), the surgeon uses a mallet (not shown) to hit strike plate 115 so as to advance shaft 85 of impactor 20 distally, whereby to seat prosthetic acetabular cup 110 within the anatomy.

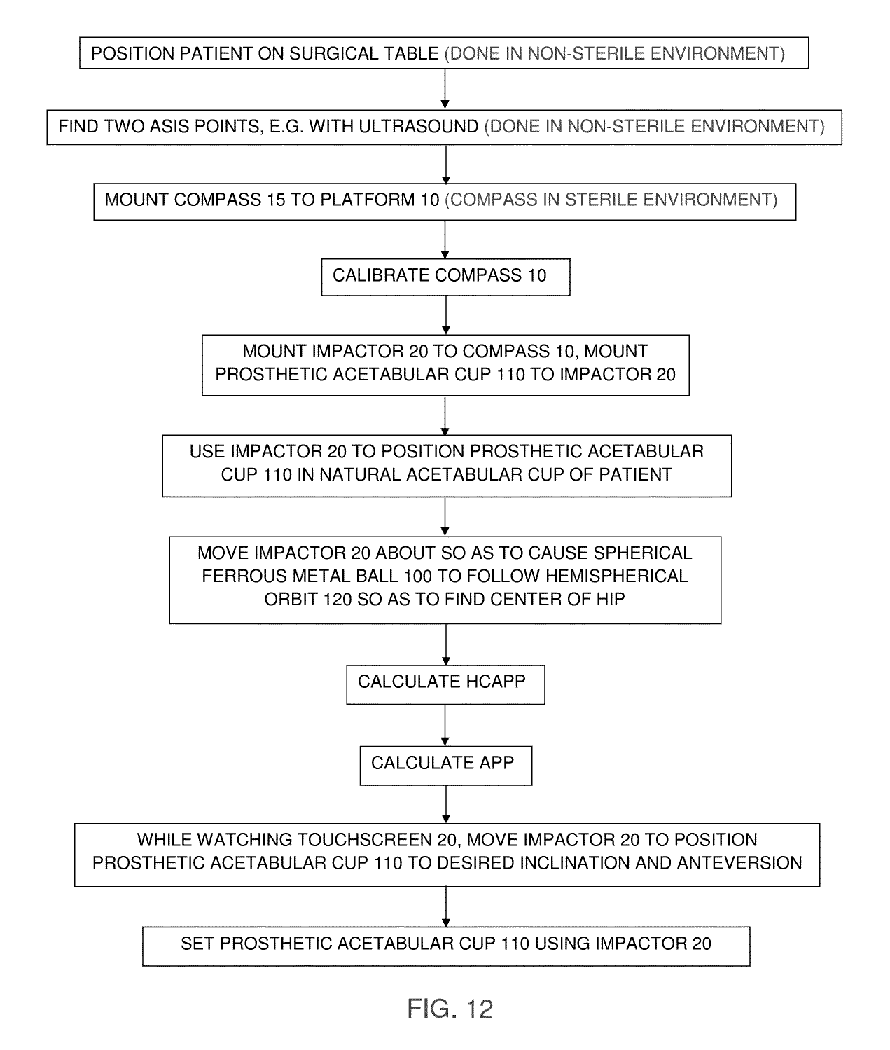

See FIG. 12, which comprises a flowchart illustrating operation of the present invention.

### Exemplary Procedure

1. Set the patient on a surgical table in the lateral position, with the patient held in place by the patient positioner throughout the procedure (e.g., via pads, clamps, etc.). The patient positioner is mounted to the surgical table. The patient positioner preferably comprises active transducers of an ultrasound device which are located in the two anterior pads of the patient positioner that are to be placed over the anterior/superior iliac spines (ASIS) of the pelvis. Platform 10 of computer-guided system 5 is mounted to the patient positioner. Note that this portion of the procedure takes place in a non-sterile environment.

2. Use the ultrasound device (i.e., the two active transducers which are mounted to the patient positioner) to find the two anterior/superior iliac spine (ASIS) points relative to the patient positioner (and hence relative to platform 10). Again, note that this portion of the procedure takes place in a non-sterile environment.

If desired, other means may be used to find the position of the two ASIS points relative to the patient position (and platform 10), e.g., a digitizer.

3. Mount compass 15 to platform 10 by disposing spherical ferrous metal ball 60 of first arm 35 in magnetic spherical recess 25 formed in platform 10, whereby to establish the aforementioned stable magnetic ball mount 61. This is done after a plastic sheet or cover (not shown) is positioned over platform 10, with this plastic sheet or cover separating the sterile environment (e.g., compass 15, impactor 20, prosthetic acetabular cup 110, etc.) from the non-sterile environment (e.g., platform 10, appropriately-programmed computer 118, etc.). Touchscreen 32 can be touch-accessed through, and viewed through, the plastic sheet or cover. Note also that the plastic sheet or cover does not interfere with the stable magnetic ball mount 61 established between compass 15 and platform 10, allowing the stable magnetic ball mount 61 to articulate. Calibrate the system by positioning concave magnetic mount 80 of compass 15 onto calibration hemisphere 30 of platform 10. Then mount impactor 20 to compass 15 by positioning concave magnetic mount 80 of compass 15 onto spherical ferrous metal ball 100 of impactor 20, whereby to establish the aforementioned stable magnetic ball mount 105. Align impactor 20 to the center of the hip joint (see above) and use computer-guided system 5 to calculate the center of the hip joint relative to the patient positioner. Note that stable magnetic ball mount 61, angular joint 75 and stable magnetic ball mount 105 all articulate as needed to accommodate this movement.

4. After the surgeon knows the center of the hip joint (from computer-guided system 5), and the two ASIS points (from ultrasound), computer-guided system 5 has all three points needed to identify the hip center anterior superior spine plane (HCAPP).

5. Computer-guided system 5 then uses the HCAPP to calculate the calculated anterior pelvic plane (APP), since the relationship of the HCAPP to the anterior pelvic plane (APP) is known. Computer-guided system 5 can then apply this natural offset of the HCAPP from the anterior pelvic plane (APP) to calculate the calculated anterior pelvic plane (APP) from the determined HCAPP. Computer-guided system 5 then displays the current disposition of impactor 20 relative to the calculated anterior pelvic plane (APP), with the current inclination and anteversion of impactor 20 being shown in a digital readout on touchscreen 32 in the context of inclination and anteversion to the calculated anterior pelvic plane (APP).

6. Now the surgeon uses impactor 20 (tracked via computer-guided system 5 relative to the calculated APP) to adjust the position of the prosthetic acetabular cup 110, by watching touchscreen 32 located on platform 10 which shows the degree of inclination and anteversion of the prosthetic acetabular cup 110 to the calculated anterior pelvic plane (APP) to the surgeon in real time as the surgeon moves impactor 20 relative to the pelvis (and hence moves prosthetic acetabular cup 110 relative to the pelvis).

7. When the surgeon has achieved the desired inclination and anteversion of prosthetic acetabular cup 110, the surgeon hits strike plate 115 of impactor 20 with a mallet so as to set the prosthetic acetabular cup in the pelvis.

It should be appreciated that the elegance of present invention is that it allows the surgeon to perform the surgical procedure in substantially the same manner traditionally utilized when setting the prosthetic acetabular cup “by eye”, except that the surgeon instead has access to highly accurate critical positional data while setting prosthetic acetabular cup 110 in the native acetabulum using the impactor, e.g., by watching touchscreen 32 on platform 10 which accurately shows the surgeon the precise current degree of inclination and anteversion of the prosthetic acetabular cup relative to the calculated anterior pelvic plane (APP). Thus, the present invention is completely compatible with existing surgical technique and simply provides the surgeon with an easy-to-use and reliable measurement system which confirms that the prosthetic acetabular cup 110 is being set in accordance with the surgeon's pre-surgical determination (e.g., “42 degrees inclination, 20 degrees anteversion” relative to the anterior pelvic plane (APP)).

### Compass with Two Spherical Encoders 62

In another form of the present invention, concave magnetic mount 80 (set at distal end 70 of second arm 40 of compass 15) and spherical ferrous metal ball 100 of impactor 20 may comprise a spherical encoder 62 (e.g., an optical spherical encoder, a magnetic spherical encoder, a mechanical spherical encoder, etc.) which is capable of reporting the disposition of spherical ferrous metal ball 100 (and hence the disposition of impactor 20) relative to second arm 40 of compass 15. In this form of the invention, the angle between second arm 40 of compass 15 and shaft 85 of impactor 20 can be determined, since the spherical encoder 62 can reliably provide data regarding the orientation of impactor 20.

In view of this, and in view of the fact that the spherical encoder 62 disposed at the junction of compass 15 and platform 10 can provide the disposition of first arm 35 of compass 15 relative to platform 10, and in view of the fact that angular sensor 76 can provide the angle between first arm 35 and second arm 40, computer-guided system 5 can then determine the disposition of impactor 20 (or another tool carried at the distal end of compass 5) relative to platform 10, and hence the disposition of impactor 20 (or another tool carried at the distal end of compass 5) relative to patient positioner 21. Since platform 10 is secured to patient positioner 21, and since the patient is secured to patient positioner 21, this arrangement allows the disposition of impactor 20 (or another tool carried at the distal end of compass 5) to be determined relative to the patient.

### Compass with Accelerometer

In another preferred form of the present invention, compass 15 comprises an accelerometer 125 (shown schematically in FIGS. 7, 8 and 11—note that while accelerometer 125 is shown on the distal end of second arm 40 in FIGS. 7, 8 and 11, accelerometer 125 could also be positioned on first arm 35 or both first arm 35 and second arm 40). This accelerometer can detect the mallet strikes on impactor 20, which then allows computer-guided system 5 to record the degree of inclination and anteversion of the prosthetic acetabular cup 110 at the time that the prosthetic acetabular cup 110 is set. This form of the invention also provides additional positioning information, obtained at a high data acquisition rate, to computer-guided system 5. In addition, this construction provides a degree of redundancy for the spherical encoder 62 disposed at the base of compass 15 (i.e., the spherical encoder 62 disposed at the magnetic ball mount 61 at the junction of platform 10 and compass 15).

Alternatively, if desired, the spherical encoder 62 at the base of compass 15 can be omitted, and compass 15 can be equipped with only angular sensor 76 and accelerometer 125—in this case, two compasses 15 (each carrying an accelerometer) are used to connect impactor 20 to platform 10, which yields enough information for computer-guided system 5 to determine the orientation of impactor 20 vis-à-vis the pelvis (and hence identify the center of the hip).

### Other Uses for Compass 15

In the hip application discussed above, compass 15 can utilize just one spherical encoder 62 (i.e., at the base of compass 15) and can use a “dumb” magnetic spherical joint to connect compass 15 to impactor 20, since this gives computer-guided system 5 enough information to find the center of the hip—this is because the position of impactor 20 is restrained by centering prosthetic acetabular cup 110 in the natural acetabular cup as impactor 20 is moved about, so that the geometry to be solved is simplified, and one spherical encoder 62 (and angular sensor 76) provides adequate information for computer-guided system 5.

However, compass 15 can also serve as a full-service digitizer, by simply providing a passive tip at the distal end of compass 15 (rather than a concave magnetic mount 80 at the distal end of compass 15 for mounting impactor 20 to compass 15).

Furthermore, with two spherical encoders 62 (i.e., one at the base of compass 15 and one at the joinder of compass 15 with impactor 20), compass 15 will provide sufficient information to calculate the position of the tip of any surgical tool attached to the distal end of compass 15, i.e., without requiring that the surgical tool be pivoted about a “socket” in the manner discussed above with respect to impactor 20. Thus, compass 15 can act as a tracker for the tip of the surgical tool, and hence can be used to guide use of the surgical tool. This may be utilized where the surgical tool is a manual tool or where the surgical tool is mounted to a robotic arm (i.e., compass 15 can be used to guide the robotic arm). Thus it will be appreciated that where compass 15 is provided with two spherical encoders 62, compass 15 can be used as a tracker for a any surgical tool mounted on the distal end of compass 15, e.g., a cutting instrument such as the cutting instrument offered by Blue Belt Technologies.

Alternatively, where compass 15 is provided with only one spherical encoder 62, and where it is desired to use compass 15 to support a probe which will act as a digitizer, the probe can simply be mounted to two compasses 15 (each having only one spherical encoder 62), in which case the data provided by the two compasses 15 will provide enough information to track the probe (e.g., so that it can act as a digitizer) or act as an instrument tracker.

In addition, where compass 15 is used to support a surgical instrument using IMU tracking technology, compass 15 will make the IMU tracking technology more robust because it can compensate for any drift or errors in the IMU tracking technology.

### Knee Application

Ideally, when doing the femoral side of a knee replacement, one would like to use a custom cutting jig (e.g., such as a rapid 3D printed cutting jig) to appropriately cut the distal femur—the use of a custom cutting jig saves the surgeon time, allows less experienced surgeons to safely and efficiently perform the procedure, eliminates the need to provide expensive instrumentation for the surgeon, etc.

To make the custom cutting jig, it is important to know the geometry of distal femur and also the center of rotation of the hip.

Significantly, compass 15 can be used to facilitate creation of the custom cutting jig. This may be done in the following manner:

1. Put a fully-functional, but miniature, platform 10 on the distal femur.

2. In this form of the invention, platform 10 comprises an accelerometer, and may comprise an inertial measurement unit (IMU) of the sort comprising an accelerometer and a gyroscope.

3. Use compass 15 as a digitizer to map the surface of the distal femur.

4. Move the leg—this action moves platform 10 (which is attached to the leg), so that the inertial measurement unit (IMU) on the platform enables computer-guided system 5 to identify the center of rotation of the hip.

Thus, by using platform 10 and compass 15 to identify the center of the hip and to digitize the distal surface of the femur, a more accurate intraoperative cutting jig (either custom or adjustable) can be provided.

There are a number of commercial advantages to using compass 15 to create the custom cutting jig: (i) it is extremely accurate, inexpensive, has 3 degrees-of-freedom articulation and is removable; (ii) it is accurate due to sphere geometry being controlled to 25 millionths of a mm, (iii) it is low cost (and hence disposable); (iv) it uses a magnetic mount (so it is removable and re-attachable); (v) using magnetic mounts (different geometries and polarity) make surgical errors in instrument use nearly impossible; (vi) the sensors effortlessly follow surgical flow without surgeon input; and (vii) the system is intuitive enough that only a modest educational program is necessary for successful use of the instrument.

In another form of the present invention, an adjustable cutting jig may be used in place of a custom cutting jig. More particularly, in this form of the invention, the adjustable cutting jig is adjusted according to 3D information provided by compass 15 functioning as a sterile digitizer tool with knowledge of the hip center and detailed distal femur anatomy.

### Anterior Hip Surgery

Anterior hip surgery is becoming increasingly popular. However, with an anterior approach, it can be difficult to determine the two ASIS points using ultrasound due to the location of the incision relative to the ultrasound pads. In this situation, compass 15 can be used in its digitizer mode to identify the two ASIS points, and then attached to impactor 20 to determine the center of the hip joint, thereby identifying the three points needed to find the HCAPP (i.e., ASIS-ASIS-hip center), whereupon the aforementioned cup positioning approach can be used.

### Modifications

It should also be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A computer-guided system for determining the disposition of an object, the computer-guided system comprising:
a platform;
a compass removably and adjustably mounted to the platform, the compass comprising:
a first arm having a proximal end and a distal end;
a second arm having a proximal end and a distal end;
the proximal end of the first arm being removably and adjustably mounted to the platform by a magnetic ball mount, wherein the magnetic ball mount comprises a spherical encoder;
the proximal end of the second arm being movably mounted to the distal end of the first arm by a pivot mount, wherein the pivot mount comprises an angular sensor; and
determining means for determining the disposition of the distal end of the second arm relative to the platform by using data from the spherical encoder and the angular sensor.

2. A computer-guided system according to claim 1 wherein the magnetic ball mount comprises a ball mounted to the proximal end of the first arm and a recess formed in the platform, and further wherein at least one of the ball and the platform comprises a magnet and the other of the ball and the platform comprises a material attracted to the magnet.

3. A computer-guided system according to claim 2 wherein a sterile divider is disposed over the platform and extends between the ball mounted to the proximal end of the first arm and the recess formed in the platform.

4. A computer-guided system according to claim 1 wherein the determining means comprises an appropriately-programmed computer.

5. A computer-guided system according to claim 4 wherein the spherical encoder and the angular sensor are wirelessly connected to the appropriately-programmed computer.

6. A computer-guided system according to claim 1 wherein the object is mounted to the distal end of the second arm and the computer-guided system determines the orientation of the object relative to the platform.

7. A computer-guided system according to claim 2 wherein the object is removably and adjustably mounted to the distal end of the second arm by a magnetic ball mount.

8. A computer-guided system according to claim 7 wherein the magnetic ball mount comprises a recess formed in the distal end of the second arm and a ball mounted to the object, and further wherein at least one of the recess formed in the second arm and the ball comprises a magnet and the other of the recess formed in the second arm and the ball comprises a material attracted to the magnet.

9. A computer-guided system according to claim 6 wherein the determining means are configured to determine the disposition of the object relative to the platform by using data from the spherical encoder and the angular sensor as the distal end of the second arm is moved in a hemispherical orbit about a point.

10. A computer-guided system according to claim 1 wherein the object comprises a surgical tool.

11. A computer-guided system according to claim 10 wherein the surgical tool comprises an impactor for setting a prosthetic acetabular cup.

12. A computer-guided system according to claim 7 wherein the magnetic ball mount between the distal end of the second arm and the object comprises a spherical encoder.

13. A computer-guided system according to claim 1 further comprising an accelerometer mounted to the compass.

14. A computer-guided system according to claim 13 further comprising an inertial measurement unit (IMU) mounted to the compass.

15. A computer-guided system according to claim 1 wherein the platform is secured to a patient positioner, so that the platform is fixed relative to the patient.

16. A method for determining the disposition of an object, the method comprising:
providing a computer-guided system for determining the disposition of an object, the computer-guided system comprising:
a platform;
a compass removably and adjustably mounted to the platform, the compass comprising:
a first arm having a proximal end and a distal end;
a second arm having a proximal end and a distal end;
the proximal end of the first arm being removably and adjustably mounted to the platform by a magnetic ball mount, wherein the magnetic ball mount comprises a spherical encoder;
the proximal end of the second arm being movably mounted to the distal end of the first arm by a pivot mount, wherein the pivot mount comprises an angular sensor; and
determining means for determining the disposition of the distal end of the second arm relative to the platform by using data from the spherical encoder and the angular sensor;
mounting the object to the distal end of the second arm; and
using the computer-guided system to determine the orientation of the object relative to the platform.

17. A method according to claim 16 wherein the magnetic ball mount comprises a ball mounted to the proximal end of the first arm and a recess formed in the platform, and further wherein at least one of the ball and the platform comprises a magnet and the other of the ball and the platform comprises a material attracted to the magnet.

18. A method according to claim 17 wherein a sterile divider is disposed over the platform and extends between the ball mounted to the proximal end of the first arm and the recess formed in the platform.

19. A method according to claim 16 wherein the determining means comprises an appropriately-programmed computer.

20. A method according to claim 19 wherein the spherical encoder and the angular sensor are wirelessly connected to the appropriately-programmed computer.

21. A method according to claim 16 wherein the object is removably and adjustably mounted to the distal end of the second arm by a magnetic ball mount.

22. A method according to claim 21 wherein the magnetic ball mount comprises a recess formed in the distal end of the second arm and a ball mounted to the object, and further wherein at least one of the recess formed in the second arm and the ball comprises a magnet and the other of the recess formed in the second arm and the ball comprises a material attracted to the magnet.

23. A method according to claim 21 wherein the determining means are configured to determine the disposition of the object relative to the platform by using data from the spherical encoder and the angular sensor as the distal end of the second arm is moved in a hemispherical orbit about a point.

24. A method according to claim 16 wherein the object comprises a surgical tool.

25. A method according to claim 24 wherein the surgical tool comprises an impactor for setting a prosthetic acetabular cup.

26. A method according to claim 21 wherein the magnetic ball mount between the distal end of the second arm and the object comprises a spherical encoder.

27. A method according to claim 16 further comprising an accelerometer mounted to the compass.

28. A method according to claim 27 further comprising an inertial measurement unit (IMU) mounted to the compass.

29. A method according to claim 16 wherein the platform is secured to a patient positioner, so that the platform is fixed relative to the patient.

30. A method for setting a prosthetic acetabular cup in the native acetabular cup with a desired inclination and anteversion, the method comprising:
providing a computer-guided system for determining the orientation of the prosthetic acetabular cup, the computer-guided system comprising:
a platform;
a compass removably and adjustably mounted to the platform, the compass comprising:
a first arm having a proximal end and a distal end;
a second arm having a proximal end and a distal end;
the proximal end of the first arm being removably and adjustably mounted to the platform by a magnetic ball mount, wherein the magnetic ball mount comprises a spherical encoder;
the proximal end of the second arm being movably mounted to the distal end of the first arm by a pivot mount, wherein the pivot mount comprises an angular sensor; and
determining means for determining the disposition of the distal end of the second arm relative to the platform by using data from the spherical encoder and the angular sensor;
determining the two ASIS points;
determining the center of the hip using the computer-guided system;
determining the HCAPP using the two ASIS points and the center of the hip;
determining the calculated APP using the HCAPP;
mounting the prosthetic acetabular cup to the distal end of the second arm; and
using the computer-guided system to set the prosthetic acetabular cup in the native acetabular cup with a desired inclination and anteversion.

31. A computer-guided system for determining the disposition of an object, the computer-guided system comprising:
a platform;
an object;
a first compass removably and adjustably mounted to the platform, the first compass comprising: a first arm having a proximal end and a distal end; a second arm having a proximal end and a distal end; the proximal end of the first arm being removably and adjustably mounted to the platform by a magnetic ball mount; the proximal end of the second arm being movably mounted to the distal end of the first arm by a pivot mount, wherein the pivot mount comprises an angular sensor; an accelerometer mounted to at least one of the first arm and the second arm; wherein the distal end of the second arm is mounted to the object;
a second compass removably and adjustably mounted to the platform, the second compass comprising: a first arm having a proximal end and a distal end; a second arm having a proximal end and a distal end; the proximal end of the first arm being removably and adjustably mounted to the platform by a magnetic ball mount; the proximal end of the second arm being movably mounted to the distal end of the first arm by a pivot mount, wherein the pivot mount comprises an angular sensor; an accelerometer mounted to at least one of the first arm and the second arm; wherein the distal end of the second arm is mounted to the object; and
determining means for determining the disposition of the relative to the platform by using data from the angular sensor and accelerometer of the first compass and data from the angular sensor and accelerometer of the second compass.
